(19) United States
(12) Patent Application Publication
(10) Pub. No.: US 2024/0284940 A1
(43) Pub. Date: Aug. 29, 2024

(54) Crumble feed formulation for ruminant animals and method for preparing said crumble feed formulation

(71) Applicant: GrainCorp Limited, Sydney (AU)

(72) Inventors: Bruce Burgess, Sydney (AU); Sara Labaf, Sydney (AU); Saeedeh Taheri, Sydney (AU)

(21) Appl. No.: 18/572,315

(22) PCT Filed: Jul. 5, 2022
(86) PCT No.: PCT/AU2022/050699

§ 371 Date: Dec. 20, 2023

(30) Foreign Application Priority Data
Jul. 5, 2021 (AU) 2021902027

(51) Int. Cl. A23K 50/10; A23K 10/30; A23K 20/10; A23K 20/158; A23K 20/163; A23K 40/10

(52) U.S. Cl. CPC A23K 50/10; A23K 10/30; A23K 20/10; A23K 20/158; A23K 20/163; A23K 40/10

(57) ABSTRACT

A crumble feed formulation for reducing methane production by a ruminant animal, said crumble feed formulation is disclosed. The crumble feed formulation comprises an effective amount of at least one species of red marine macroalgae combined within a crumble nutrient, wherein said crumble feed formulation has a dry matter content of from about 85% to about 95% by weight.

### FIELD OF THE INVENTION

The present invention relates to a crumble feed formulation for ruminant animals, and a method for preparing said crumble feed formulation.

The invention has been developed primarily for use as a crumble feed formulation to be fed to ruminant animals either directly or as part of a feed ration to reduce enteric fermentation in said ruminant animals, thereby reducing the subsequent production of the greenhouse gas (GHG), methane (CH₄), and will be described hereinafter with reference to this application.

### BACKGROUND OF THE INVENTION

Methane (CH₄) in the atmosphere is a highly potent greenhouse gas (GHG) with a reported global warming potential 28 times greater than carbon dioxide (CO₂). Agriculture is a major contributor to the global GHG inventory and ruminant enteric fermentation is the largest agricultural source responsible for 60% of agriculture's contributions, primarily as methane.

The anti-methanogenic properties of using many types of seaweeds (macroalgae) as feed additives has now been confirmed by many researchers. Bromoform is the bioactive ingredient responsible for the anti-methanogenesis in seaweed. However, bromoform is a volatile chemical that can be easily lost to the atmosphere if not kept in a stable environment.

One particular genus of seaweed is the red marine macroalgae, Asparagopsis spp. It has been demonstrated that when Asparagopsis spp. seaweed is fed to ruminant livestock, it delivers a significant reduction in the production of methane, as well as gains in productivity.

To maintain the anti-methanogenic activity of Asparagopsis spp. seaweed, the seaweed biomass is typically processed by freeze-drying to remove nearly all of the moisture. Unfortunately, however, freeze dried Asparagopsis spp. is unpalatable to livestock.

To address this issue, it has been commonplace to add the freeze dried Asparagopsis spp. to a crumble nutrient. However, this has had a significant impact on the formulation of the crumble nutrient.

More recently, it has been demonstrated that an alternative process to freeze-drying involves homogenising freshly collected Asparagopsis spp. in oil.

The present invention thus seeks to provide a crumble feed formulation for ruminant animals, and a method for preparing said crumble feed formulation, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to a first aspect of the present invention there is provided a crumble feed formulation for reducing methane production by a ruminant animal, said crumble feed formulation comprising an effective amount of at least one species of red marine macroalgae combined within a crumble nutrient, said crumble feed formulation having a dry matter content of from about 85% to about 95% by weight.

Preferably, the species of red marine macroalgae is an Asparagopsis spp. selected from the group consisting of Asparagopsis taxiformis and Asparagopsis armata.

In one embodiment, the crumble feed formulation has a dry matter content of from about 88% to about 95% by weight.

In one embodiment, the species of red marine macroalgae is freeze dried.

In one embodiment, the species of red marine macroalgae comprises an anti-methanogenic agent.

Suitably, the anti-methanogenic agent is bromoform.

In one embodiment, the bromoform is present in the crumble feed formulation in an amount from about 100 mg/kg to about 30,000 mg/kg by weight.

Preferably, the crumble nutrient comprises water.

In one embodiment, the water is present in the crumble feed formulation in an amount from about 1% to about 10% by weight.

Preferably, the crumble nutrient comprises a carbohydrate source.

In one embodiment, the carbohydrate source is present in the crumble feed formulation in an amount of about 5% to about 25% by weight.

More preferably, the carbohydrate source comprises a sugar-based carbohydrate source.

In one embodiment, the sugar-based carbohydrate source comprises molasses and/or glycerine.

Preferably, the crumble nutrient comprises an edible oil selected from the group consisting of canola oil, sunflower oil, safflower oil, soybean oil, and any combination thereof.

In one embodiment, the edible oil is present in the crumble feed formulation in an amount from about 1% to about 15% by weight.

In one embodiment, the edible oil comprises canola oil.

Preferably, the crumble feed formulation further comprises one or more excipients or additives selected from the group consisting of wetting agents, thickeners, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen), protein meals (cottonseed meal, canola meal, soyabean meal) and medications.

In one embodiment, the effective amount of the at least one species of red marine macroalgae combined within the crumble nutrient is from about 2% to about 90% by weight.

In one embodiment, the crumble nutrient comprises:
(a) about 1% to about 10% by weight of water;
(b) about 1% to about 15% by weight of an edible oil; and
(c) about 5% to about 25% by weight of a carbohydrate source.

In one embodiment, the crumble nutrient comprises:
(a) about 60% to about 85% by weight of canola meal; and
(b) about 5% to about 30% by weight of a carbohydrate source.

Preferably, the edible oil is canola oil and the carbohydrate source is molasses.

According to a second aspect of the present invention there is provided a crumble feed formulation for reducing methane production by a ruminant animal, said crumble feed formulation comprising an effective amount of at least one species of red marine macroalgae combined within a crumble nutrient comprising:
(a) about 1% to about 10% by weight of water;
(b) about 1% to about 15% by weight of an edible oil; and
(c) about 5% to about 25% by weight of a carbohydrate source,
wherein the crumble feed formulation has a dry matter content of from about 85% to about 95% by weight.

According to a third aspect of the present invention there is provided a crumble feed formulation for reducing methane production by a ruminant animal, said crumble feed formulation comprising an effective amount of at least one species of red marine macroalgae combined within a crumble nutrient comprising:
(a) about 60% to about 85% by weight of canola meal; and
(b) about 5% to about 30% by weight of a carbohydrate source,
wherein the crumble feed formulation has a dry matter content of from about 85% to about 95% by weight.

In one embodiment, the species of red marine macroalgae is immersed in an edible oil.

Preferably, the edible oil selected from the group consisting of canola oil, sunflower oil, safflower oil, soybean oil, and any combination thereof.

In one embodiment, the species of red marine macroalgae is Asparagopsis taxiformis.

In one embodiment, the effective amount of Asparagopsis taxiformis combined within the crumble nutrient is from about 2% to about 90% by weight.

In one embodiment, the effective amount of Asparagopsis taxiformis combined within the crumble nutrient is from about 1% to about 20% by weight.

Suitably, the Asparagopsis taxiformis comprises an anti-methanogenic agent in the form of bromoform.

In one embodiment, the bromoform is present in the crumble feed formulation in an amount from about 100 mg/kg to about 30,000 mg/kg by weight.

In one embodiment, the crumble feed formulation has a dry matter content of from about 88% to about 95% by weight.

According to a third aspect of the present invention there is provided a method of feeding a ruminant animal, the method comprising feeding the crumble feed formulation of the first or second aspect to a ruminant animal.

Preferably, the crumble feed formulation is mixed with animal feed.

In one embodiment, the crumble feed formulation is mixed with the animal feed at a ratio of about 0.1% to about 5% relative to the combined weight of the crumble feed formulation and the animal feed.

In one embodiment, the animal feed comprises a feedstock selected from the group consisting of dry animal fodder, straw, hay, alfalfa, grains, forage, grass, fruits, vegetables, oats, crop residue, fats and oils and any combination thereof.

Preferably, said ruminant animal is cattle, sheep or goat.

According to a fourth aspect of the present invention there is provided a method for reducing methane production by a ruminant animal, said method comprising the step of administering to said animal a crumble feed formulation comprising an effective amount of at least one species of red marine macroalgae combined within a crumble nutrient, said crumble feed formulation having a dry matter content of from about 85% to about 95% by weight.

Preferably, the method further comprises the step of mixing the crumble feed formulation with animal feed.

In one embodiment, the crumble feed formulation is mixed with the animal feed at a ratio of about 0.1% to about 5% relative to the combined weight of the crumble feed formulation and the animal feed.

In one embodiment, the animal feed comprises a feedstock selected from the group consisting of dry animal fodder, straw, hay, alfalfa, grains, forage, grass, fruits, vegetables, oats, crop residue, fats and oils and any combination thereof.

Preferably, said ruminant animal is cattle, sheep or goat.

According to a fifth aspect of the present invention there is provided a method of producing a crumble feed formulation for reducing methane production by a ruminant animal, the method comprising the step of combining an effective amount of at least one species of red marine macroalgae within a crumble nutrient to form a crumble feed formulation having a dry matter content of from about 85% to about 95% by weight.

Preferably, the species of red marine macroalgae is an Asparagopsis spp. selected from the group consisting of Asparagopsis taxiformis and Asparagopsis armata.

In one embodiment, the crumble feed formulation has a dry matter content of from about 88% to about 95% by weight

In one embodiment, the species of red marine macroalgae is freeze dried.

Preferably, the crumble nutrient comprises:
(a) about 1% to about 10% by weight of water;
(b) about 1% to about 15% by weight of an edible oil; and
(c) about 5% to about 25% by weight of a carbohydrate source.

In one embodiment, the species of red marine macroalgae is immersed in an edible oil.

Preferably, the edible oil selected from the group consisting of canola oil, sunflower oil, safflower oil, soybean oil, and any combination thereof.

In a preferred embodiment, the crumble nutrient comprises:
(a) about 60% to about 85% by weight of canola meal; and
(b) about 5% to about 30% by weight of a carbohydrate source,
wherein the crumble feed formulation has a dry matter content of from about 85% to about 95% by weight.

In one embodiment, the species of red marine macroalgae comprises an anti-methanogenic agent.

Suitably, the anti-methanogenic agent is bromoform.

In one embodiment, the bromoform is present in the crumble feed formulation in an amount from about 100 mg/kg to about 30,000 mg/kg by weight.

In one embodiment, the method further comprises the step of introducing one or more excipients or additives to the crumble nutrient.

Preferably, the one or more excipients or additives are selected from the group consisting of wetting agents, thickeners, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen), protein meals (cottonseed meal, canola meal, soyabean meal) and medications.

According to a sixth aspect of the present invention there is provided a crumble feed formulation for reducing methane production by a ruminant animal, the crumble feed formulation prepared according to the method of the fifth aspect.

Other aspects of the invention are also disclosed.

### BRIEF DESCRIPTION OF THE DRAWINGS

Preferred embodiments of the invention will now be described, by way of example only, with reference to the accompanying drawings, in which:

FIG. 1 shows a schematic representation for the environmental and packaging condition and time intervals for stability analysis of bromoform in certain feed formulations prepared according to a preferred embodiment of the present invention;

FIG. 2 and FIG. 3 show plots showing bromoform concentrations in feedlot concentrate (Example 3) at different conditions during a 12-week period of analysis; The dashed line shows the expected amount of bromoform in the feed;

FIG. 4 shows the percentage loss of bromoform for feedlot concentrate (Example 3) at different conditions during the 12-week period of analysis (This is calculated based on the concentrations quantified at time 0 (no storage));

FIG. 5 and FIG. 6 show plots showing Bromoform concentration (mg/g) in basic crumble feed formulation (Example 1) at different conditions during an 8-week period of analysis; error bars are ±SD (n=2), concentrations with an asterisk are significantly less than day 0 with p<0.05 based on comparison with the Fisher LSD method; and

FIG. 7 shows the percentage loss of bromoform for basic crumble feed formulation (Example 1) at different conditions during the 8-week period of analysis (This is calculated based on the concentrations quantified at time 0 (no storage)).

### DETAILED DESCRIPTION OF SPECIFIC EMBODIMENTS

It should be noted in the following description that like or the same reference numerals in different embodiments denote the same or similar features.

The present invention is predicated on the finding that Asparagopsis spp., whether it be freeze-dried or fully immersed in oil, can be incorporated into a molasses/vegetable oil/canola meal-based crumble nutrient to render it palatable and acceptable to livestock, while maintaining a sufficient level of bromoform to facilitate the desired anti-methanogenesis activity for reducing the amount of methane produced by the ruminant animal.

This molasses/vegetable oil/canola meal-based crumble feed formulation can be fed to ruminant animals in various forms, either as part of a mixed feed ration or directly to the animal. In either form, the crumble feed formulation supplements the animal's requirements for nutrients such as energy, protein, trace minerals and vitamins, as well as delivering various medications. Those fed directly to ruminant animals can be formulated to varying levels of palatability to control intake. Those mixed in a feed ration improve the palatability of the ration, reduce dust and separation, as well as providing an even distribution of the nutrients/components supplied.

The inventors have successfully demonstrated that the crumble feed formulation can be stored for a period of time, while maintaining good consistency without the components separating from the crumble feed formulation.

In this respect, combining Asparagopsis spp. with a molasses/vegetable oil/canola meal-based crumble nutrient enables the delivery of the Asparagopsis spp. seaweed to ruminant animals in a stable, palatable form, utilizing an existing supply chain, while maintaining a sufficient amount of bromoform within the Asparagopsis spp. to facilitate the anti-methanogenesis activity.

### Definitions

As used herein, the term “reducing” includes the reduction of amount of substance in comparison with a reference. For example, the reduction in the amount of total methane produced by a ruminant animal or animals administered a crumble feed formulation comprising a red marine macroalgae according to the present invention, relative to an animal or animals not administered the crumble feed formulation of the present invention. The reduction can be measured in vitro with an artificial rumen system that simulates anaerobic fermentation, or in vivo with animals confined in respiration chambers. It is within the knowledge and skill of those trained in the art to assess enteric methanogenesis by a ruminant animal.

As used herein, the term “reducing methane production” refers to the reduction of methane produced in the animal's digestive system. The term includes the specific volume of methane generated as a result of anaerobic fermentation, for example, in the systems described herein. Fermentation in the rumen and the gut of a ruminant gives rise to production of methane. The present invention aims to reduce this process, such as to reduce the total amount of methane produced in the animal's digestive system. It is within the knowledge and skill of those trained in the art to assess methane production by a ruminant animal.

As used herein, the term “effective amount”, is used to indicate a quantity of at least one species of red marine macroalgae sufficient to allow improvement, e.g., reduction in the amount of methane production in comparison with a reference or control. Within the meaning of the present invention, the methane reductive effect can be measured in the rumen with an artificial rumen system, or by in vivo oral administration to ruminants.

As used herein, the terms “administer” and “administered”, are used to indicate the action of introducing at least one species of red marine macroalgae according to the present invention into the ruminant animal's digestive system. More particularly, this administration is an administration by oral route. This administration can in particular be carried out by supplementing a feed ration intended for the ruminant animal with the crumble feed formulation of the present invention, the thus supplemented feed ration then being ingested by the ruminant animal. The administration can also be carried out using a stomach tube or any other means making it possible to directly introduce the red marine macroalgae into the animal's digestive system.

As used herein, the term “ruminant”, is used to indicate an herbivorous mammal of the suborder Ruminantia; animals that are able to acquire nutrients from plant-based food by fermenting it in a specialized stomach prior to digestion, principally through microbial action. For the purpose of describing the present invention, said ruminant animal is cattle, sheep or goat.

As used herein, the term “homogenising”, is used to indicate the breakup the red marine macroalgae biomass to facilitate release of at least one anti-methanogenic agent from the biomass. The homogenising can take place by, for example, crushing, grinding, milling, blending, cutting, slicing, dicing, or any combination thereof.

As used herein, the term “shelf life” is used to indicate that the amount or level of an anti-methanogenic agent in the crumble feed formulation according to the embodiments of the present invention is not significantly reduced for the period of time and temperature that the crumble feed formulation is stored at. In other words, the amount of time elapsed before there is a significant reduction in the level of the anti-methanogenic agent is taken to indicate the shelf life. The term “stability” is also used as an indicator of shelf life. The ‘stability’ of the anti-methanogenic agent can be assessed by measuring the level of the anti-methanogenic agent over time.

As used herein, “dry matter (DM) content” is used to indicate the percent of the crumble feed formulation of the present invention that is not water, assuming that all the moisture from the crumble feed formulation has been removed.

As used herein, “as-fed” is used to indicate the percent of the crumble feed formulation of the present invention in which none of the moisture has been removed.

### Crumble Feed Formulation

A crumble feed formulation for reducing methane production by a ruminant animal according to a preferred embodiment of the present invention, will now be described.

There are two recognised species of Asparagopsis present throughout the world, one is a tropical/sub-tropical (Asparagopsis taxiformis) type and the other is a temperate (Asparagopsis armata) type. In either case, both species comprise an anti-methanogenic agent in the form of bromoform. It is this agent that is responsible for reducing enteric fermentation in ruminant animals.

According to the embodiments of the present invention described herein, the Asparagopsis species used in the preparation of the crumble feed formulations is Asparagopsis taxiformis.

However, it will be appreciated by persons skilled in the relevant art that the crumble feed formulations according to the present invention may also be prepared using Asparagopsis armata.

Asparagopsis taxiformis and Asparagopsis armata are harvested from the sea as a biomass. Once harvested, these species are then typically processed by freeze drying or immersing in an edible oil such as a vegetable oil in order to retain as much of the anti-methanogenic agent, bromoform as possible.

What follows therefore is a description of a method for preparing crumble feed formulations for ruminant animals in which the Asparagopsis taxiformis is provided in a freeze-dried or oil extract form.

### Freeze Dried Asparagopsis

The Asparagopsis taxiformis biomass is freeze dried according to standard freeze-drying procedures.

The quantity of bromoform in the freeze-dried Asparagopsis taxiformis is typically between about 2 mg/g and about 40 mg/g according to the dry matter (DM) weight.

According to one embodiment of the present invention, the bromoform level in the freeze-dried Asparagopsis taxiformis used to prepare the crumble feed formulation of Example 1 (see Table 1) was calculated to be 6.5 mg/g of freeze dried seaweed.

In this embodiment, the crumble feed formulation is prepared by combining the freeze-dried Asparagopsis taxiformis within a crumble nutrient that comprises in its basic form, water, a carbohydrate source, and an edible oil.

The carbohydrate source is a sugar-based carbohydrate source selected from the group consisting of molasses and glycerine.

The edible oil is a vegetable oil selected from the group consisting of canola oil, sunflower oil, safflower oil, soybean oil, and any combination thereof.

According to this embodiment, the crumble nutrient for combining with the freeze-dried Asparagopsis taxiformis in, comprises from:
(a) about 1% to about 10% by weight of water;
(b) about 1% to about 15% by weight of canola oil; and
(c) about 5% to about 25% by weight of molasses.

In this embodiment, the effective amount of freeze-dried Asparagopsis taxiformis combined within the crumble nutrient is determined to be from about 2% to about 90% by weight. This would equate to a bromoform content in the resulting crumble feed formulation, measured immediately after preparation, of from about 100 mg/kg to about 30,000 mg/kg by weight.

The inventors have determined that the crumble feed formulation of Example 1 in Table 1 has a formulated bromoform level of 4875 mg/kg (as fed) or 5395 mg/Kg (DM).

The crumble feed formulation prepared according to this embodiment has an average dry matter content of from about 85% to about 95% by weight.

### Excipients/Additives

According to some embodiments, the crumble feed formulation of the present invention may further comprise one or more excipients that are necessary to prepare the desired feed form and/or one or more additives aimed at improving the quality of the crumble feed formulation.

The one or more excipients or additives may be selected from the group consisting of wetting agents, thickeners, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen), protein meals (cottonseed meal, canola meal, soyabean meal) and medications.

### EXAMPLES

What follows is a basic crumble feed formulation comprising freeze-dried Asparagopsis taxiformis formulated for feeding to a ruminant animal.

In this embodiment, the bromoform level in the basic crumble feed formulation may range from approximately 2,000 mg/kg to 30,000 mg/kg (as fed).

### Example 1: Preparation of a Basic Crumble Feed Formulation Comprising Freeze-Dried Asparagopsis taxiformis

TABLE 1
Basic Crumble Feed Formulation

| Component | % By Weight | Mixing Time (min) |
|---|---|---|
| Asparagopsis taxiformis | 75.0 | 0 |
| Water | 5.0 | 0 |
| Molasses | 14.0 | 0 |
| Canola oil | 6.0 | 4 |

In a preferred embodiment, one or more of the excipients or additives described above are introduced successively to the basic crumble feed formulation in the order of addition in which they appear in Tables 2 and 3 (see below).

What follows is a sample dry loose mix pasture feed supplement comprising freeze-dried Asparagopsis taxiformis formulated to be fed directly to a ruminant animal at a rate of 1 kg per day, supplying true protein and minerals, with the option of adding vitamins and medications.

### Example 2: Preparation of a Pasture Feed Supplement Comprising Freeze-Dried Asparagopsis taxiformis

TABLE 2
Pasture Feed Supplement

| Component | % By Weight | Mixing Time (min) |
|---|---|---|
| Asparagopsis taxiformis | 4.2 | 0 |
| Water | 2.0 | 0 |
| Molasses | 15.0 | 0 |
| Canola oil | 6.0 | 4 |
| Dicalcium Phosphate (DCP) 18%^{a} | 1.0 | 0 |
| Salt (fine dried) | 2.5 | 0 |
| Trace Mineral Premix^{b} | 0.25 | 0 |
| Copper 12.7%^{c} | 0.005 | 1 |
| Canola Meal | 69.045 | 2 |

^{a}Dicalcium Phosphate 18% (supplied by BEC Feed Solutions).
^{b}The trace mineral premix (manufactured and supplied by Rabar Pty Ltd) comprises zinc (12%), manganese (4%), cobalt (0.177%), iodine (1.178%), selenium (0.265%) and salt (51.17%).
^{c}SQM ™ Copper (manufactured and supplied by Qualitech, Inc.).

In this embodiment, the bromoform level in the pasture fed supplement is approximately 273 mg/kg (as fed) or 312 mg/Kg (DM).

It will be understood by persons skilled in the relevant art that the pasture fed supplement can be formulated to feed to a ruminant animal at a higher rate (2 to 3 kg per day), although this would result in a lower bromoform concentration.

What follows is a sample dry loose mix feedlot concentrate comprising freeze-dried Asparagopsis taxiformis formulated to be mixed in a feed ration for feeding to a ruminant animal at a rate of 3% DM.

### Example 3: Preparation of a Feedlot Concentrate Comprising Freeze-Dried Asparagopsis taxiformis

TABLE 3
Feedlot Concentrate

| Component | % By Weight | Mixing Time (min) |
|---|---|---|
| Asparagopsis taxiformis | 11.6 | 0 |
| Water | 2.0 | 0 |
| Molasses | 4.0 | 0 |
| Canola oil | 2.0 | 4 |
| Ammonium Sulfate | 5.0 | 0 |
| Urea | 25.0 | 1 |
| Salt (fine dried) | 6.67 | 0 |
| Trace Mineral Premix^{a} | 0.67 | 0 |
| Copper 12.7%^{b} | 0.033 | 0 |
| Monensin 400^{c} | 0.1834 | 0 |
| Magnesium Carbonate | 4.0 | 1 |
| Vitamin A 40%^{d} | 0.0447 | 0 |
| Vitamin E 75%^{e} | 0.034 | 1 |
| Limestone (fine) | 38.7649 | 2 |

^{a}The trace mineral premix (manufactured and supplied by Rabar Pty Ltd) comprises zinc (12%), manganese (4%), cobalt (0.177%), iodine (1.178%), selenium (0.265%) and salt (51.17%).
^{b}SQM ™ Copper (manufactured and supplied by Qualitech, Inc.).
^{c}Monensin 400 (manufactured and supplied by Nutriment Health, Phibro Animal Health or International Animal Health).
^{d}Rovimix ™ A 400 DLC (manufactured and supplied by DSM Animal Nutrition & Health).
^{e}Rovimix ™ E 750 DLC Liquid (manufactured and supplied by DSM Animal Nutrition & Health).

In this embodiment, the bromoform level in the feedlot concentrate is approximately 750 mg/kg (as fed).

### Food Ration

The molasses/vegetable oil/canola meal-based crumble feed formulations prepared according to embodiments of the present invention can be fed to ruminant animals in various forms, either as part of a mixed feed ration or directly to the animal.

In either form, the crumble feed formulation supplements the animal's requirements for nutrients such as energy, protein, trace minerals and vitamins, as well as delivering various medications. Those fed directly to ruminant animals can be formulated to varying levels of palatability to control intake. Those mixed in a feed ration improve the palatability of the ration, reduce dust and separation, as well as providing an even distribution of the nutrients/components supplied.

What follows is a description of a method of preparing a feed ration based on the crumble feed formulation of the embodiments of the present invention in combination with a feedstock for use in feeding to a ruminant animal such as cattle, sheep or goat.

The feedstock may be selected from the group consisting of dry animal fodder, straw, hay, alfalfa, grains such as wheat, barley, sorghum wheat), roughage (hay, straw, silage), protein meals (cotton seed meal, canola meal), high protein seeds (cotton seed, lupins, chickpeas), forage, grass, fruits, vegetables, oats, crop residue, fats and oils and any combination thereof.

Typically, the feedlot concentrate is mixed with the feedstock at a ratio of about 0.1% to about 5% relative to the combined weight of the feedlot concentrate and the feedstock.

### Example 4: Preparation of a Feed Ration

A typical feed ration for feeding to a ruminant animal to reduce methane production comprises: 0% to 90% grain (wheat, barley, sorghum etc.), 2% to 95% roughage (hay, straw, silage), 0% to 12% protein meals (cotton seed meal, canola meal), 0% to 12% high protein seeds (cotton seed, lupins, chickpeas), 0% to 4% oil and a 0.1% to 5% concentration of the feedlot concentrate (Example 3) described above.

What follows is a sample starter feed ration based on the feedlot concentrate described in Example 3 (see Table 3) in which the feedlot concentrate is mixed with the feedstock at a ratio of about 2.6% relative to the combined weight of the feedlot concentrate and the feedstock.

TABLE 4
Starter Feed Ration

| Component | % By Weight |
|---|---|
| Oaten hay | 33.4 |
| Sorghum silage | 20.0 |
| Barley 12.0 | 20.0 |
| Wheat 12.0 | 10.0 |
| Cereal straw | 6.0 |
| Canola meal 38.0 | 4.0 |
| Cotton seed | 4.0 |
| Feedlot concentrate (Example 3) | 2.6 |

The starter feed ration in Table 4 provides a formulated bromoform level of 19.60 mg/kg (as fed) or 25.60 mg/Kg (Dry Matter for a feed ration with a dry matter content of 76.59%). This equates to the high level used in the Asparagopsis feedlot feeding trial conducted by the Commonwealth Scientific and Industrial Research Organisation (CSIRO) on behalf of the Meat & Livestock Australia Limited (Published: 31 May 2018 (see: https://www.mla.com.au/research-and-development/reports/2020/asparagopsis-feedlot-feeding-trial/).

What follows is a sample finisher feed ration based on the feedlot concentrate described in Example 3 (see Table 3) in which the feedlot concentrate is mixed with the feedstock at a ratio of about 2.6% relative to the combined weight of the feedlot concentrate and the feedstock.

TABLE 5
Finisher Feed Ration

| Component | % By Weight |
|---|---|
| Barley 12.0 | 45.9 |
| Sorghum silage | 20.0 |
| Wheat 12.0 | 20.0 |
| Cereal straw | 6.0 |
| Cotton seed | 4.0 |
| Feedlot concentrate (Example 3) | 2.6 |
| Vegetable oil (blend) | 1.5 |

The finisher feed ration in Table 5 provides a formulated bromoform level of 19.60 mg/kg (as fed) or 25.23 mg/Kg (Dry Matter for a feed ration with a dry matter content of 77.70%). This also equates to the high level used in the CSIRO trial.

### Bromoform Stability Analysis

What follows is a study aimed to evaluate the stability of the main bioactive compound of the seaweed (bromoform) after it has been supplemented into different feed formulations.

The study involved: adding freeze-dried Asparagopsis taxiformis to different feed formulations kept at different environmental conditions; assessing the stability of bromoform during the time and evaluating the effect of ingredients on bromoform stability in a feed formulation in which the base component of the formulation is molasses.

This study was devised to cover as many commercial conditions as possible for the stability analysis of bromoform in the feed formulations.

### Materials and Method

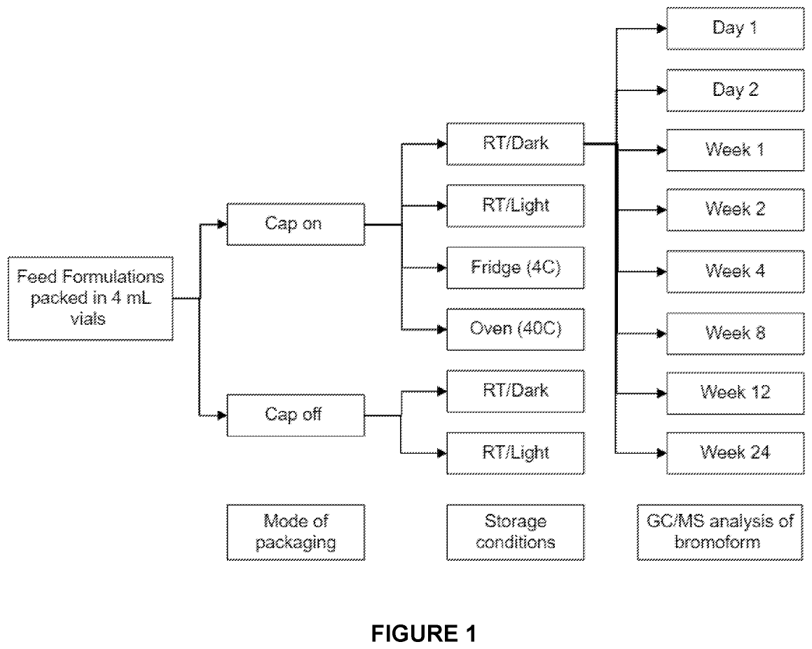

Two formulations, namely basic crumble feed formulation (Example 1) and feedlot concentrate (Example 3) were prepared as represented in Table 6. The feed formulations were packed in 4-ml glass vials with two replicates for each condition and time interval and placed at different conditions as shown in FIG. 1. The vials were taken out and analysed for their bromoform content after each time interval.

The bromoform analysis was performed using an internal method. The interval of testing was as follows: on day 1, day 2, week 1, week 2, week 4, week 8, and week 12. Extractions and analysis were performed in duplicate due to the high accuracy of the method and the GC/MS instrument used, which led to very low variance in replicates. The limited number of replicates was due to a large number of samples and conditions along with the limitation in the availability of some of the raw materials.

TABLE 6
Feed Formulations

% By Weight

| Component | Example 3 | Example 1 |
|---|---|---|
| Water | 2 | 5 |
| Xanthan gum | — | — |
| Canola oil | 2 | 6 |
| Ammonium sulfate | 5 | — |
| Urea | 25 | — |
| Salt (fine dried) | 6.67 | — |
| Trace Mineral Premix^{a} | 0.67 | — |
| Copper 12.7%^{b} | 0.03 | — |
| Monensin 400^{c} | 0.18 | — |
| Magnesium Carbonate | 4 | — |
| Vitamin A 40%^{d} | 0.03 | — |
| Vitamin E 75%^{e} | 0.04 | — |
| Limestone (fine) | 38.76 | — |
| Asparagopsis taxiformis^{f} | 11.6 | 75 |
| Molasses | 4 | 14 |

^{a}The trace mineral premix (manufactured and supplied by Rabar Pty Ltd) comprises zinc (12%), manganese (4%), cobalt (0.177%), iodine (1.178%), selenium (0.265%) and salt (51.17%).
^{b}SQM ™ Copper (manufactured and supplied by Qualitech, Inc.).
^{c}Monensin 400 (manufactured and supplied by Nutriment Health, Phibro Animal Health or International Animal Health).
^{d}Rovimix ™ A 400 DLC (manufactured and supplied by DSM Animal Nutrition & Health).
^{e}Rovimix ™ E 750 DLC Liquid (manufactured and supplied by DSM Animal Nutrition & Health).
^{f}freeze dried.

### Analysis of Bromoform Using GCMS

Bromoform was extracted by sonicating 100 mg of homogenized feed with 10 mL of high-performance liquid chromatography-grade methanol, for 30 min at 4° C. The sample were then centrifuged at 3000 g for 10 min, methanol was removed and transferred into a clean 50 mL Falcon tub. The extraction was repeated using 10 mL of methanol for 30 min at 4° C. in sonication bath. After centrifugation, 20 mL methanol were further diluted using methanol. The methanol phase was collected and analysed by GC-MS as described below.

Gas chromatography was performed using an Agilent 8890 [Agilent, Australia] gas chromatograph equipped and a polyethylene glycol-coated phase on a polyimide-coated fused silica capillary column (INNOWAX 19091N1331, 30 m, 0.25 mm i.d., 0.25 μM). All injections (1 μl) were performed in the splitless mode. The inlet liner (Agilent 5190-2293, splitless, single taper, glass wool) was replaced after 50 samples. The injection port was held at 180° C. and the GC-MS interface at 280° C. The GC was held at 40° C. for 1 min, ramped at 16° C. min-1 to 250° C., then held at this temperature for 2 min. Helium was used as the carrier gas. Mass spectrometry was performed on an Agilent 7010 QQQ. Ions characteristic of the standards and target compounds were monitored in the multiple reaction monitoring (MRM) mode. Target compound standards were run at the start of each sample set and at regular intervals within sample sets. Target compound concentration in each sample was calculated from the peak areas. This area was then converted to concentration by reference to standard curves. Masses of target compounds are expressed as mass compound per unit algal dry weight. Commercial standards used were bromoform (Sigma-Aldrich).

### Statistical Analysis

A general linear model was fit to the data of each feed formulation and ANOVA (analysis of variance) was performed. Data of different conditions and time intervals were compared using the Fisher LSD method with a 95% confidence level. Statistical analyses were performed in Excel or Minitab 2020.

### Results and Discussion

### Stability of Bromoform in Different Feed Formulations

### Feedlot Concentrate (Example 3)

The result of the stability tests of bromoform content in the feedlot concentrate (Example 3) is represented in Error! Reference source not found. The statistical analysis was not performed on the data of this feed formulation due to high standard deviations among the data. These high standard deviations could be the results of the very inhomogeneous nature of the feed formulation, which was caused by a high proportion of granular urea content. Increasing the number of replicates or sample size did not reduce the standard deviation (data not shown), and therefore, it is suggested that the urea should be used in the powdered format in this formulation in future studies.

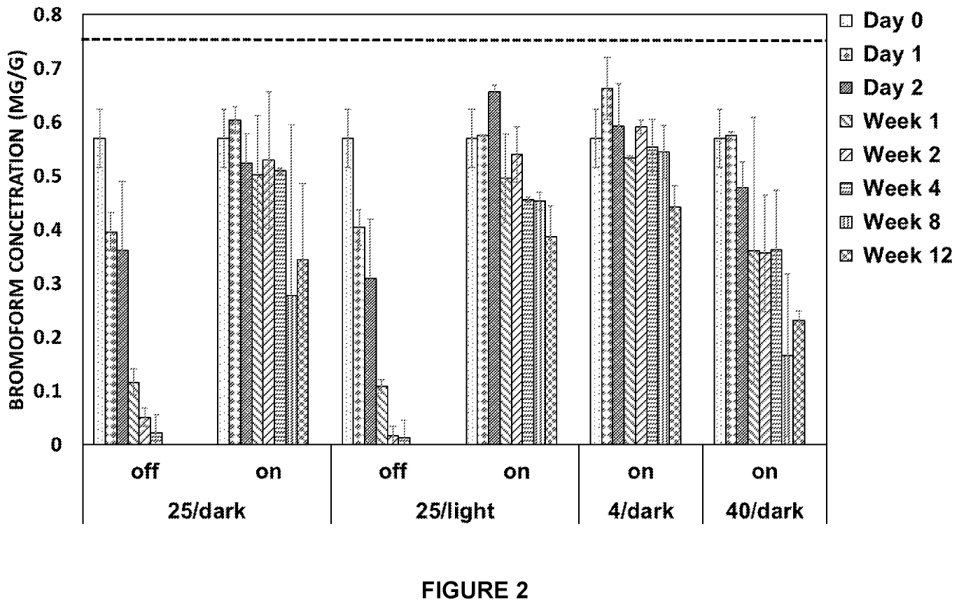

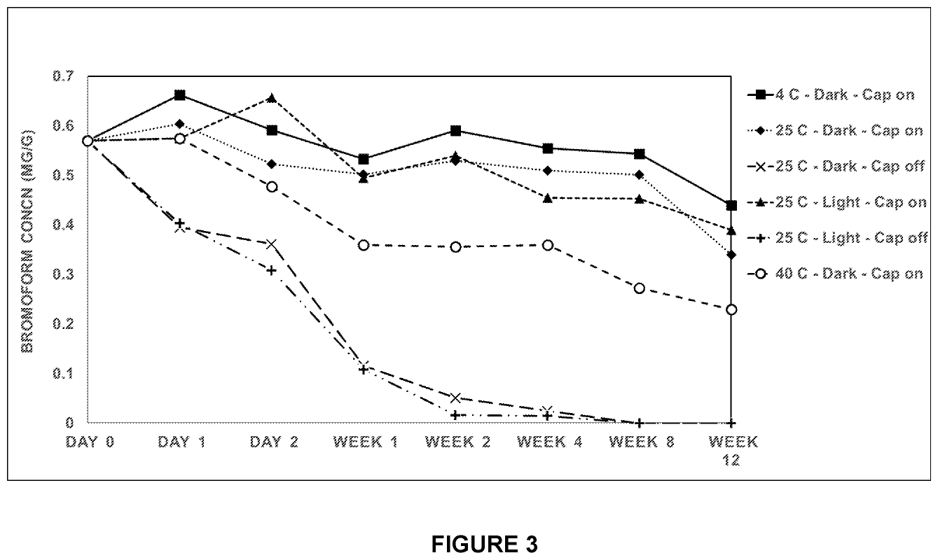

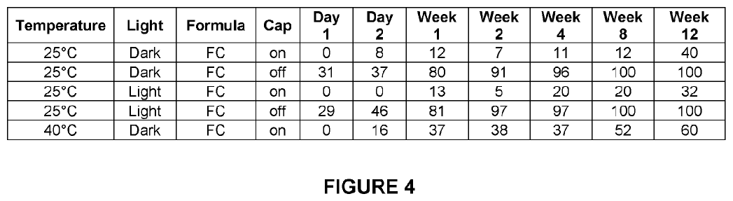

In general, the plots in FIGS. 2 and 3 show that the bromoform concentration reduces from 0.57 mg/g at day 0 to 0.39 and 0.31 mg/g of feed after 1 day of storage at room temperature with exposure to air (uncapped) under dark and light conditions, respectively. These amounts were accordingly reduced to 0.02 and 0.012 mg/g of feed after 4 weeks. Storage of the feed formulations at 40° C. reduced the bromoform content from 0.57 mg/g to about 0.47 and 0.2 mg/g after 2 days and 12 weeks. The inventors observed that around 29% of bromoform is lost during preparation of the sample at day 0 (FIG. 4). The feed concentrate is almost stable during 8 weeks of storage at 4° C. There is slight decrease on the concentration of bromoform for the samples stored at room temperature over time (in average 20%). Exposure to air had the biggest effect on the concentration of bromoform at the end of the 8-week period, while exposure to light had an insignificant effect. There is almost no bromoform left in the feed concentrate after 2 weeks for samples stored at 25° C. and exposed to air (FIG. 4). The inventors speculated that some of the ingredients of the feed formulation may undergo an interaction with bromoform. This hypothesis is investigated using a liquid formulation. The effect of compounds including nitrogen-based and/or metal ions was investigated (data not shown).

### Basic Crumble Feed Formulation (Example 1)

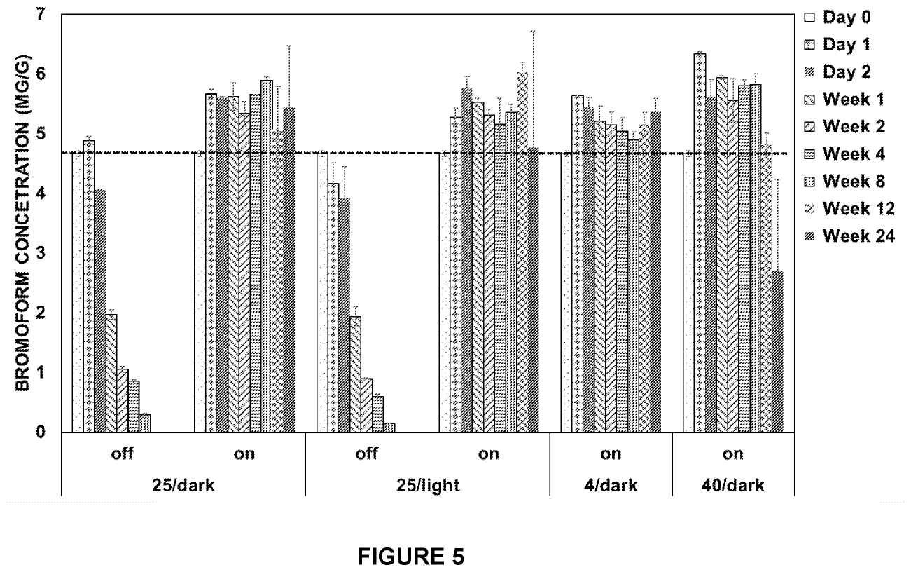

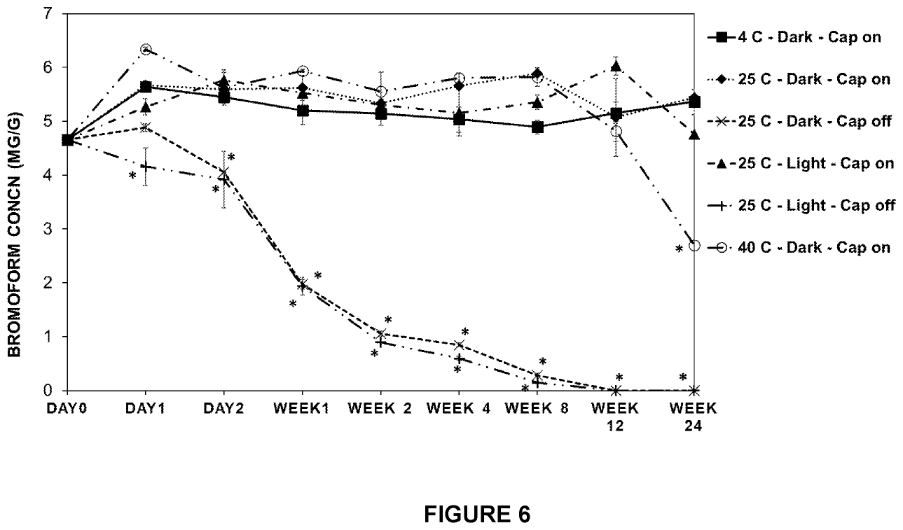

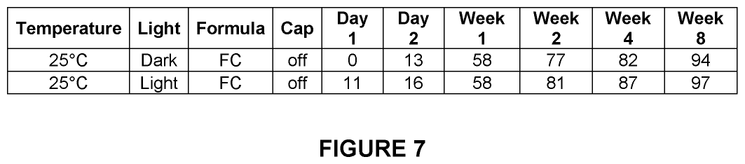

FIGS. 5, 6 and 7 show the results of bromoform stability in the basic crumble feed formulation (Example 1). A general linear model was fit to the data (R-sq=99.4%, R-sq(adj)=98.5%) and a post hoc Fisher LSD method comparison was performed, which clearly shows that there was no reduction in the bromoform concentrations of the feed formulation in any of the keeping conditions up to week 8 except for the samples exposed to air (uncapped). The bromoform content was observed to decrease from 4.66 mg/g to about 4 mg/g for the uncapped samples, both under dark and light conditions after 2 days, and reached below 1 mg/g at week 4. The bromoform content of the feed formulation stored at 40° C. was observed to decrease to below 3 mg/g after 6 months of storage. Therefore, the inventors consider that a shelf life of 6 months could be recommended for the basic crumble feed formulation stored at room temperature and 3 months at 40° C. in an airtight container.

### Discussion

The feedlot concentrate with 11.6% Asparagopsis taxiformis (Example 3) was very inhomogeneous due to the presence of a large amount of granular urea. This caused a larger fluctuation and therefore large standard deviations in the results.

Increasing the sample replicates of this particular feed formulation to four or increasing the initial amount of the sample to extract bromoform did not help to reduce the standard deviations. Therefore, no statistical analysis was performed on the results for this feed formulation. A glance at the plots in FIGS. 2 and 3, however, revealed that exposing this particular feed formulation to air (uncapped) dramatically reduced the bromoform content, even after one day, and the degradation in bromoform content continued to reach to almost 100% after 2 weeks.

Exposure to air was observed to be destructive to the bromoform content of the basic crumble feed formulation (Example 1) too. Indeed, the bromoform content was found to reduce significantly after one day under light conditions, and after 2 days under dark conditions when exposed to air.

Notably, however, temperature and light conditions did not affect bromoform stability in the crumble feed formulation up to week 8 of the analysis. Therefore, this feed formulation is found to have good bromoform stability at higher temperatures.

It was expected that the bromoform concentration in these feed formulations would be the same as the bromoform content of the added seaweed according to the law of conservation of mass. This expectation can be fulfilled when there are no chemical reactions in the matrix of the feed formulation. However, it was observed that the bromoform concentrations were significantly less than the bromoform content of the added seaweed in the feedlot concentrate (Example 3) studied (Dashed black line in FIG. 2)). Based on this observation, the inventors speculated that there might be interactions between bromoform and one or more of the components in the feed formulations.

The result of evaluating the effect of certain component(s) in the feed formulation on bromoform stability confirmed that those components containing nitrogen compounds and/or metal ions could accelerate the degradation of bromoform in the feed formulation (data not shown).

Based on these observations, it is considered that feed formulations comprising Asparagopsis taxiformis should be devoid of such compounds if the aim is to keep these feed formulations under harsh temperature conditions or for a long period of time.

### Conclusion

Based on the results of this study, it is considered that freeze-dried Asparagopsis taxiformis can be added to a feed formulation comprising molasses as a base provided that no components containing nitrogen or metal ions are introduced into the feed formulation. This is to make sure that the feed formulation has a long shelf life for industrial applications.

Therefore, one solution is that the freeze dried Asparagopsis taxiformis is added to the base without additives (molasses, water, gum, and oil for liquid feed) and the additives are mixed separately. These two separate premixed bases could be mixed in the farm at the time of feeding.

### Oil Immersed Asparagopsis

The Asparagopsis taxiformis biomass is homogenised in an edible oil according to the method outlined in the literature [M. Magnusson et al. Algal Research, 51 (2020) 102065]. The oil is then separated from the Asparagopsis taxiformis after the bromoform has been extracted from the seaweed into the oil.

In this embodiment, the edible oil is canola oil. Other vegetable oils suitable for this purpose may also include sunflower oil, safflower oil, soybean oil, and any combination thereof.

The quantity of bromoform in the oil immersed Asparagopsis taxiformis is calculated to be between about 1000 mg/kg and about 40,000 mg/kg according to the dry matter (DM) weight.

Once homogenised, the resulting oil immersed Asparagopsis taxiformis is then mixed with a crumble nutrient that comprises in its basic form, canola meal, and a carbohydrate source.

The carbohydrate source is a sugar-based carbohydrate source selected from the group consisting of molasses and glycerine.

In a preferred embodiment, the crumble nutrient for mixing with about 1% to about 20% of the oil immersed Asparagopsis taxiformis, comprises from:
(a) about 60% to about 85% by weight of canola meal; and
(b) about 5% to about 30% by weight of molasses.

Again, the inventors have found that it is preferable that these components are added in the order of addition in which they appear in Table 6 (see below).

For instance, the canola meal and oil immersed Asparagopsis taxiformis are combined, prior to mixing with the carbohydrate source (molasses).

In this embodiment, the effective amount of oil immersed Asparagopsis taxiformis mixed within the crumble nutrient is from about 1% to about 20% by weight. This equates to a bromoform content in the resulting crumble feed formulation, measured immediately after preparation, of from about 50 mg/kg to about 8000 mg/kg by weight.

The crumble feed formulation prepared according to this embodiment has an average dry matter content of from about 85% to about 95% by weight.

In much the same way as the crumble feed formulations comprising the freeze-dried Asparagopsis taxiformis described above, the crumble feed formulation comprising oil immersed Asparagopsis taxiformis may also comprise one or more of the same excipients and/or additives described above to improve the quality of the crumble feed formulation.

What follows is a basic crumble feed formulation comprising oil immersed Asparagopsis taxiformis for use as part of a mixed feed ration for a ruminant animal or fed directly to said ruminant animal.

In this embodiment, the bromoform level in this oil-immersed basic crumble feed formulation may range from approximately 50 mg/kg to 8000 mg/kg (as fed).

### Example 5: Preparation of a Basic Crumble Feed Formulation Comprising Oil-Immersed Asparagopsis taxiformis

TABLE 8
Basic Crumble Feed Formulation^{a}

| Component | % By Weight | Mixing Time (min) |
|---|---|---|
| Canola meal | 73.0 | 0 |
| Asparagopsis taxiformis^{b} | 7.0 | 10 |
| Molasses | 20.0 | 20 |

^{a}The dry matter of the basic crumble feed formulation is 88.2%
^{b}The immersion oil is canola oil.

In a preferred embodiment, one or more of the excipients or additives described above are introduced successively to the basic crumble feed formulation in the order of addition in which they appear in Table 8.

At a daily intake of 1 kg per head, the basic crumble feed formulation in Table 8 provides a formulated bromoform level of 280 mg/kg (as fed) or 317.3 mg/Kg (for a dry matter content of 88.2%). This equates to the high level used in the Asparagopsis feedlot feeding trial conducted by the Commonwealth Scientific and Industrial Research Organisation (CSIRO) on behalf of the Meat & Livestock Australia Limited (Published: 31 May 2018 (see: https://www.mla.com.au/research-and-development/reports/2020/asparagopsis-feedlot-feeding-trial/).

In other words, when fed to adult cattle, 1 kg of the basic crumble feed formulation comprising oil-immersed Asparagopsis taxiformis, whether fed directly or as part of a mixed ration, delivers around 280 mg of bromoform per day,

### Materials and Methods

As described above, the components of each crumble feed formulation are added in the order of addition in which they appear in the corresponding table (see Tables 1-3).

By way of a guide, the following method outlines the procedure for preparing the feedlot concentrate described in Example 3 (see Table 3), for every 1000 kg's mixed.

Firstly, 116 Kg of freeze-dried Asparagopsis taxiformis is placed in the vessel of a horizontal ribbon mixer (custom built). While stirring at a rate of 20 rpm to 60 rpm, 20 Kg of water, 40 Kg of molasses and 20 Kg of Canola oil are gradually introduced and stirred for around 20 minutes to hydrate and coat the Asparagopsis taxiformis.

Once the Asparagopsis taxiformis is fully coated with the canola oil and molasses, ammonium sulfate (50 Kg) and urea (250 Kg) are then introduced into the vessel, and the mixture is stirred at a stirring rate of 20 to 60 rpm for around 5 minute, before the following components: fine dried NaCl (66.7 Kg), Tracer Mineral Premix™ (6.7 Kg), Copper 12.7% (0.33 Kg), Monensin 400 (1.834 Kg) and MgCO₃ (40 Kg), are then introduced at a stirring rate of 20 to 60 rpm for a further 5 minute. Vitamin A 40% (0.34 Kg) and Vitamin E 75% (0.447 Kg) are then added to the vessel with gradual stirring at a stirring rate of 20 to 60 rpm for a further 5 minutes, before 387.649 Kg of the last component, limestone (fine), is added at a stirring rate of 20 to 60 rpm for around 10 minutes to obtain the feedlot concentrate.

The moisture content (%) and the dry matter (DM) content of the crumble feed formulations were determined by the formulation specifications obtained using the Format ARA formulations program (supplied by Cargill Format Solutions).

The bromoform content within the Asparagopsis taxiformis starting materials, and within the crumble feed formulation itself, is determined by gas chromatography-mass spectrometry (GC-MS) using methanol extraction.

For the freeze-dried samples, bromoform was extracted by sonicating 100 mg of homogenized seaweed (using a pestle and mortar) with 10 mL of high-performance liquid chromatography-grade methanol, for 30 minutes at 4° C. The sample was then centrifuged at 3000 g for 10 minutes, and the methanol removed and transferred into a clean 50 mL Falcon tube. The extraction process was repeated using 10 mL of methanol for 30 minutes at 4° C. in a sonication bath. After centrifugation, a further 20 mL methanol was added to dilute the sample. The methanol phase was then collected and analysed by GC-MS as described below.

Gas chromatography was performed using an Agilent gas chromatograph 8890 (supplied by Agilent, Australia) equipped with a polyethylene glycol-coated phase on a polyimide-coated fused silica capillary column (INNOWAX 19091N1331, 30 m, 0.25 mm i.d., 0.25 μM). All injections (1 μl) were performed in the splitless mode. The inlet liner (Agilent 5190-2293, splitless, single taper, glass wool) was replaced after 50 samples. The injection port was held at 180° C. and the GC-MS interface at 280° C. The GC was held at 40° C. for 1 minute, ramped at 16° C. min-1 to 250° C., then held at this temperature for 2 minutes. Helium was used as the carrier gas. Mass spectrometry was performed on an Agilent 7010 triple quadrupole mass spectrometer (supplied by Agilent, Australia). Ions characteristic of the standards and target compounds were monitored in the multiple reaction monitoring (MRM) mode. Target compound standards were run at the start of each sample set and at regular intervals within sample sets. The target compound concentration in each sample was calculated from the peak area. This area was then converted to concentration by reference to standard curves. Masses of target compounds are expressed as mass compound per unit algal dry weight. Commercial standards used were bromoform (Sigma-Aldrich).

### Conclusion

The inventors have prepared crumble feed formulations comprising Asparagopsis species seaweed in either a freeze-dried or oil-immersed form, for feeding to ruminant animals. The inventors have determined that when, for instance, the freeze-dried Asparagopsis species is first coated with canola oil and/or molasses, there is a reduced likelihood of the bromoform associated with the Asparagopsis species degrading too quickly. This finding correlates with the inventors' observation that the oil-immersed Asparagopsis species maintains a high degree of bromoform from the outset. As such, the as-prepared basic crumble feed formulations, and subsequent feed rations formulated therefrom, maintain a sufficient level of bromoform to facilitate the desired anti-methanogenesis activity for reducing the amount of methane produced by the ruminant animal.

### Advantages

The crumble feed formulations described in embodiments of the present invention herein provide a number of advantages, including, but not limited to:
[1] Being able to maintain consistency (not separate) of the crumble feed formulation;
[2] Improve palatability of the Asparagopsis species; and
[3] Maintain stability of the bromoform in the crumble feed formulation.

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

Similarly, it should be appreciated that in the above description of example embodiments of the invention, various features of the invention are sometimes grouped together in a single embodiment, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

### Comprising and Including

In the claims which follow and in the preceding description of the invention, except where the context requires otherwise due to express language or necessary implication, the word “comprise” or variations such as “comprises” or “comprising” are used in an inclusive sense, i.e., to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the animal feed industry.

1. A crumble feed formulation for reducing methane production by a ruminant animal, said crumble feed formulation comprising an effective amount of at least one species of red marine macroalgae combined within a crumble nutrient, said crumble feed formulation having a dry matter content of from about 85% to about 95% by weight.

2. The crumble feed formulation according to claim 1, wherein the species of red marine macroalgae is an Asparagopsis spp. selected from the group consisting of Asparagopsis taxiformis and Asparagopsis armata.

3. The crumble feed formulation according to claim 1, wherein the crumble feed formulation has a dry matter content of from about 88% to about 95% by weight.

4. The crumble feed formulation according to claim 1, wherein the species of red marine macroalgae is freeze dried.

5. The crumble feed formulation according to claim 1, wherein the species of red marine macroalgae comprises an anti-methanogenic agent.

6. The crumble feed formulation according to claim 5, wherein the anti-methanogenic agent is bromoform.

7. The crumble feed formulation according to claim 6, wherein the bromoform is present in the crumble feed formulation in an amount from about 50 mg/kg to about 8,000 mg/kg by weight.

8. The crumble feed formulation according to claim 1, wherein the crumble nutrient comprises water.

9. The crumble feed formulation according to claim 8, wherein the water is present in the crumble feed formulation in an amount from about 1% to about 10% by weight.

10. The crumble feed formulation according to claim 1, wherein the crumble nutrient comprises a carbohydrate source.

11. The crumble feed formulation according to claim 10, wherein the carbohydrate source is present in the crumble feed formulation in an amount of about 5% to about 30% by weight.

12. The crumble feed formulation according to claim 10, wherein the carbohydrate source comprises a sugar-based carbohydrate source.

13. The crumble feed formulation according to claim 12, wherein the sugar-based carbohydrate source comprises molasses and/or glycerine.

14. The crumble feed formulation according claim 1, wherein the crumble nutrient comprises an edible oil selected from the group consisting of canola oil, sunflower oil, safflower oil, soybean oil, and any combination thereof.

15. The crumble feed formulation according claim 14, wherein the edible oil is present in the crumble feed formulation in an amount from about 1% to about 15% by weight.

16. The crumble feed formulation according claim 14, wherein the edible oil comprises canola oil.

17. The crumble feed formulation according to claim 1, further comprising one or more excipients or additives selected from the group consisting of wetting agents, thickeners, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen), protein meals (cottonseed meal, canola meal, soyabean meal) and medications.

18. The crumble feed formulation according to claim 1, wherein the effective amount of the at least one species of red marine macroalgae combined within the crumble nutrient is from about 2% to about 90% by weight.

19. The crumble feed formulation according to claim 1, wherein the crumble nutrient comprises:
(a) about 1% to about 10% by weight of water;
(b) about 1% to about 15% by weight of an edible oil; and
(c) about 5% to about 25% by weight of a carbohydrate source.

20. The crumble feed formulation according to claim 1, wherein the crumble nutrient comprises:
(a) about 60% to about 85% by weight of canola meal; and
(b) about 5% to about 30% by weight of a carbohydrate source.

21. The crumble feed formulation according to claim 19, wherein the edible oil is canola oil and the carbohydrate source is molasses.

22. A crumble feed formulation for reducing methane production by a ruminant animal, said crumble feed formulation comprising an effective amount of at least one species of red marine macroalgae combined within a crumble nutrient comprising:
(a) about 1% to about 10% by weight of water;
(b) about 1% to about 15% by weight of an edible oil; and
(c) about 5% to about 25% by weight of a carbohydrate source,
wherein the crumble feed formulation has a dry matter content of from about 85% to about 95% by weight.

23. A crumble feed formulation for reducing methane production by a ruminant animal, said crumble feed formulation comprising an effective amount of at least one species of red marine macroalgae combined within a crumble nutrient comprising:
(a) about 60% to about 85% by weight of canola meal; and
(b) about 5% to about 30% by weight of a carbohydrate source,
wherein the crumble feed formulation has a dry matter content of from about 85% to about 95% by weight.

24. The crumble feed formulation according to claim 23, wherein the species of red marine macroalgae is immersed in an edible oil.

25. The crumble feed formulation according to claim 24, wherein the edible oil selected from the group consisting of canola oil, sunflower oil, safflower oil, soybean oil, and any combination thereof.

26. The crumble feed formulation according to any one of claims 22 to 25, wherein the species of red marine macroalgae is Asparagopsis taxiformis.

27. The crumble feed formulation according to claim 22, wherein the effective amount of Asparagopsis taxiformis combined within the crumble nutrient is from about 2% to about 90% by weight.

28. The crumble feed formulation according to claim 23, wherein the effective amount of Asparagopsis taxiformis combined within the crumble nutrient is from about 1% to about 20% by weight.

29. The crumble feed formulation according to any one of claims 22 to 28, wherein the Asparagopsis taxiformis comprises an anti-methanogenic agent in the form of bromoform.

30. The crumble feed formulation according to claim 29, wherein the bromoform is present in the crumble feed formulation in an amount from about 50 mg/kg to about 8000 mg/kg by weight.

31. The crumble feed formulation according to any one of claims 22 to 30, wherein the crumble feed formulation has a dry matter content of from about 88% to about 95% by weight.

32. A method of feeding a ruminant animal, the method comprising feeding the crumble feed formulation of any one of claims 1 to 31 to a ruminant animal.

33. The method according to claim 32, wherein the crumble feed formulation is mixed with a feedstock.

34. The method according to claim 33, wherein the crumble feed formulation is mixed with the feedstock at a ratio of about 0.1% to about 5% relative to the combined weight of the crumble feed formulation and the feedstock.

35. The method according to claim 33 or 3342, wherein the feedstock is selected from the group consisting of dry animal fodder, straw, hay, alfalfa, grains, forage, grass, fruits, vegetables, oats, crop residue, fats and oils and any combination thereof.

36. The method according to claim 35, wherein said ruminant animal is cattle, sheep or goat.

37. A method for reducing methane production by a ruminant animal, said method comprising the step of administering to said animal a crumble feed formulation comprising an effective amount of at least one species of red marine macroalgae combined within a crumble nutrient, said crumble feed formulation having a dry matter content of from about 85% to about 95% by weight.

38. The method according to claim 35, further comprising the step of mixing the crumble feed formulation with a feedstock.

39. The method according to claim 38, wherein the crumble feed formulation is mixed with the feedstock at a ratio of about 0.1% to about 5% relative to the combined weight of the crumble feed formulation and the feedstock.

40. The method according to claim 38 or 39, wherein the feedstock is selected from the group consisting of dry animal fodder, straw, hay, alfalfa, grains, forage, grass, fruits, vegetables, oats, crop residue, fats and oils and any combination thereof.

41. The method according to claim 40, wherein said ruminant animal is cattle, sheep or goat.

42. A method of preparing a crumble feed formulation for reducing methane production by a ruminant animal, the method comprising the step of combining an effective amount of at least one species of red marine macroalgae within a crumble nutrient to form a crumble feed formulation having a dry matter content of from about 85% to about 95% by weight.

43. The method according to claim 42, wherein the species of red marine macroalgae is an Asparagopsis spp. selected from the group consisting of Asparagopsis taxiformis and Asparagopsis armata.

44. The method according to claim 42 or 43, wherein the crumble feed formulation has a dry matter content of from about 88% to about 95% by weight

45. The method according to any one of claims 42 to 44, wherein the species of red marine macroalgae is freeze dried.

46. The method according to claim 45, wherein the crumble nutrient comprises:
(a) about 1% to about 10% by weight of water;
(b) about 1% to about 15% by weight of an edible oil; and
(c) about 5% to about 25% by weight of a carbohydrate source.

47. The method according to any one of claims 42 to 44, wherein the species of red marine macroalgae is immersed in an edible oil.

48. The method according to claim 47, wherein the edible oil selected from the group consisting of canola oil, sunflower oil, safflower oil, soybean oil, and any combination thereof.

49. The method according to claim 47 or 48, wherein the crumble nutrient comprises:
(a) about 60% to about 85% by weight of canola meal; and
(b) about 5% to about 30% by weight of a carbohydrate source,
wherein the crumble feed formulation has a dry matter content of from about 85% to about 95% by weight.

50. The method according to any one of claims 42 to 49, wherein the species of red marine macroalgae comprises an anti-methanogenic agent.

51. The method according to claim 50, wherein the anti-methanogenic agent is bromoform.

52. The method according to claim 51, wherein the bromoform is present in the crumble feed formulation in an amount from about 100 mg/kg to about 30,000 mg/kg by weight.

53. The method according to claim 42, further comprising the step of introducing one or more excipients or additives to the crumble nutrient.

54. The method according to claim 53, wherein the one or more excipients or additives are selected from the group consisting of wetting agents, thickeners, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen), protein meals (cottonseed meal, canola meal, soyabean meal) and medications.

55. A crumble feed formulation for reducing methane production by a ruminant animal, the crumble feed formulation prepared according to the method of any one of claims 42 to 54.
